(19) United States
(12) Patent Application Publication
(10) Pub. No.: US 2011/0218216 A1
(43) Pub. Date: Sep. 8, 2011

(54) EXTENDED RELEASE PHARMACEUTICAL COMPOSITION OF DONEPEZIL

(76) Inventors: Kumaravel VIVEK, Chennai (IN); Romi Barat Singh, Varanasi (IN); Anuj Kumar Fanda, Ghaziabad (IN)

(21) Appl. No.: 13/017,395

(22) Filed: Jan. 31, 2011

(30) Foreign Application Priority Data
Jan. 29, 2010 (IN) 201/DEL/2010

(51) Int. Cl. A61K 31/445; A61P 25/28

(52) U.S. Cl. 514/319

(57) ABSTRACT

The present invention relates to an extended release pharmaceutical composition for oral administration comprising donepezil or pharmaceutically acceptable salt thereof and a release-controlling agent. Further, it relates to process for preparation of said compositions.

### FIELD OF THE INVENTION

The present invention relates to an extended-release pharmaceutical composition for oral administration comprising donepezil or a pharmaceutically acceptable salt thereof and a release-controlling agent. Further, it relates to process for preparation of said composition.

### BACKGROUND OF THE INVENTION

Donepezil is a reversible inhibitor of the enzyme acetylcholinesterase. Its main therapeutic use is in the treatment of Alzheimer's disease.

The immediate-release donepezil results in a spike in the patient's blood plasma levels within 2 hours to 5 hours after administration of the drug. The initial spike in blood plasma levels may cause undesirable side effects in patients, such as anxiety, nightmares, insomnia, and/or gastrointestinal problems.

Therefore, in recent years sustained-release formulations have been developed which would avoid a rapid increase in blood plasma concentration levels immediately after administration of the drug, thus potentially reducing or eliminating adverse side effects.

Currently, donepezil is being marketed under the trade name Aricept® by Eisai. Aricept® is available as immediate-release tablets and orally disintegrating tablets with a dose of 5 mg and 10 mg and is generally administered once per day. Also, recently Eisai has got approval from the United States Food and Drug Administration for 23 mg donepezil tablets.

Various attempts have been made in prior art to prepare sustained-release formulation of donepezil.

U.S. Patent Publication No. 2009/0208579 discloses a matrix type sustained-release preparation comprising donepezil and at least one enteric polymer. Also, it discloses a combination of enteric polymer with a water-insoluble polymer.

U.S. Patent Publication Nos. 2006/0280789 and 2007/0129402 disclose a pharmacokinetic profile of a sustained-release pharmaceutical formulation comprising donepezil and an enteric polymer. Further, it discloses a combination of enteric polymer with a water insoluble polymer.

U.S. Patent Publication No. 2008/0213368 discloses a method for stabilizing pharmaceutical composition comprising a high molecular weight basic substance and donepezil by adding a high molecular weight acidic substance to said pharmaceutical composition; wherein said high molecular weight acidic substance includes enteric polymer and high molecular weight basic substance includes water-insoluble polymer.

The prior art formulation requires the addition of an enteric polymer for preparing a stable pharmaceutical composition of donepezil. Also, the addition of an enteric polymer is required for obtaining a pH independent dissolution profile of donepezil.

The present inventors have now developed a pharmaceutical composition of donepezil, wherein the pharmaceutical composition does not comprise an enteric polymer. Hence, the present invention relates to an extended-release pharmaceutical composition for an oral administration comprising donepezil or pharmaceutically acceptable salt thereof and a release-controlling agent. The compositions, while providing a therapeutic effect over an extended period of time, also exhibit acceptable stability upon storage.

### SUMMARY OF THE INVENTION

In one general aspect, the present invention provides for an extended-release pharmaceutical composition for an oral administration consisting essentially of:
a) donepezil or a pharmaceutically acceptable salt thereof;
b) a release-controlling agent selected from the group consisting of hydrophilic polymer, hydrophobic material, hydrophobic polymer, and a mixture thereof; and
c) a microenvironment pH modifier

Embodiments of this aspect may include one or more of the following features. For example, the hydrophobic polymer is selected from the group consisting of ethylcellulose, cellulose acetate, cellulose propionate, cellulose butyrate, methacrylic acid-acrylic acid copolymers, and a mixture thereof.

The hydrophilic polymer is selected from the group consisting of polyvinylpyrrolidone, hydroxypropylcellulose, methylcellulose, hydroxypropylmethyl cellulose, polyethylene oxide, acrylic acid copolymers, and a mixture thereof.

The hydrophobic material is selected from the group consisting of hydrogenated vegetable oil, hydrogenated castor oil, carnauba wax, candellia wax, beeswax, paraffin wax, stearic acid, glyceryl behenate, cetyl alcohol, cetostearyl alcohol, and a mixture thereof.

The microenvironment pH modifier is selected form the group consisting of inorganic acid, amino acid, organic acid, and a mixture thereof.

The microenvironment pH modifier is an organic acid selected from the group consisting of lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid and salicylic acid; tosylic acid, mesylic acid, malic acid, and a mixture thereof. For example, the microenvironment pH modifier is fumaric acid and hydrophobic polymer is ethyl cellulose.

The pharmaceutical composition is in the form of tablet, capsules or granules. For example, the tablet is in the form of matrix.

The pharmaceutical composition does not include an enteric polymer.

### DETAILED DESCRIPTION OF THE INVENTION

The present inventors have now successfully developed an extended-release composition of donepezil comprising a release-controlling agent, wherein said formulation would provide the desired release profile of donepezil.

The term “pharmaceutically acceptable salts”, as used herein, includes organic or inorganic acid salt of donepezil, e.g., hydrochlorides, sulfates, acetates, phosphates, carbonates, mesylates, tartrates, citrates and tosylates.

Donepezil may be present alone or in combination with other anti-dementia drugs such as NMDA receptor antagonists (e.g., memantine), choline uptake enhancers (e.g., MKC-231), somatostatin release enhancers (e.g., FK960), neurotransmitter regulators (e.g., nefiracetam), muscarinic M1 receptor agonists (e.g., talsaclidine), benzodiazepine receptor partial inverse agonists (e.g., S-8510), and acetylcholine/noradrenaline release enhancers (e.g., T-588, T-817MA).

The term “extended release pharmaceutical composition”, as used herein, includes any pharmaceutical composition that achieves the slow-release of drug over an extended period of time, and includes prolonged, sustained, and controlled-release compositions.

The term “release controlling agent” refers to one or more of hydrophilic polymers, hydrophobic polymers, hydrophobic materials and mixtures thereof, which control the rate of release. These do not include enteric release polymers. The release-controlling agent may be present in an amount of about 25%-70% by weight of the composition.

Suitable hydrophilic polymers may include water-soluble polymers as well as water swellable polymers, such as, polyvinylpyrrolidone, hydroxypropylcellulose, hydroxypropylmethyl cellulose, methylcellulose, polysaccharides (such as, alginate, xanthan gum.), polyethylene oxide, acrylic acid copolymers (such as carbomer), and a mixture thereof.

Suitable hydrophobic polymers may include cellulose ethers, such as, ethylcellulose, propylcellulose, ethylmethylcellulose, ethylpropylcellulose, isopropylcellulose, butylcellulose; cellulose aralkyl ethers, such as benzyl cellulose; cellulose cyanoalkyl ethers such as cyanoethyl cellulose, cyanomethyl cellulose, cyanoethylmethyl cellulose, cyanopropyl cellulose), methacrylic acid-acrylic acid copolymers (e.g., Eudragit® RS, Eudragit® RL, Eudragit® NE, Eudragit® RSPO, Eudragit® RLPO) and a mixture thereof. For example, ethyl cellulose may be used as a hydrophobic polymer. It may be present in the composition as intragranular, as well as, extragranular. The ethyl cellulose may be present in a total amount of about 25%-70% by weight of the composition.

Suitable hydrophobic materials may include hydrogenated vegetable oil, hydrogenated castor oil, carnauba wax, candellia wax, beeswax, paraffin wax, stearic acid, glyceryl behenate, cetyl alcohol, cetostearyl alcohol, and a mixture thereof.

Further, the extended-release pharmaceutical composition may also include microenvironment pH modifiers, such as inorganic acids, amino acids and organic acids.

Examples of organic acids include acetic acid, benzoic acid, lauric acid, myristic acid, palmitic acid, stearic acid, oxalic acid, malonic acid, succinic acid, adipic acid, sebacic acid, fumaric acid, maleic acid, glycyrrhizic acid, glycyrrhetic acid and sorbic acid; hydroxy acids such as glycolic acid, lactic acid, malic acid, tartaric acid, citric acid, sodium dihydrogen citrate, gluconic acid and salicylic acid; sulfonic acids such as tosylic acid, mesylic acid, and malic acid. The organic acid may be present in an amount of about 15%-25% by total weight of the composition.

Examples of inorganic acids include hydrochloric acid, sulfuric acid, nitric acid, boric acid, phosphoric acid, sodium dihydrogen phosphate and potassium dihydrogen phosphate.

Examples of acidic amino acids include aspartic acid, glutamic acid, glutamic acid hydrochloride, histidine hydrochloride and glycine hydrochloride, cysteine hydrochloride and methionine.

Other microenvironment pH modifiers include citrate buffer, ascorbic acid, and BHT.

The microenvironment pH modifier may be added directly in the extended-release pharmaceutical composition comprising donepezil. Alternatively, prior to formulating pharmaceutically acceptable excipients with donepezil, the excipients may be treated with microenvironment pH modifiers, e.g., granulating excipients with aqueous or non aqueous dispersion of microenvironment pH modifiers.

Pharmaceutical composition, as used herein, may be either in the form of a matrix type extended-release preparation or a coated type extended-release preparation.

In a matrix type preparation, donepezil and the release-controlling agent are distributed uniformly in the formulation. The matrix type preparation may be in the form of tablets, granules and capsules. Further, tablets may be a layered tablet wherein one layer may comprise donepezil or a pharmaceutical acceptable salt thereof and a release-controlling agent and another layer may comprise a release-controlling agent which may act as a support layer.

Also, one layer may be immediate-release and the other layer extended-release. Capsules may comprise one or more mini tablets, and/or granules.

In a coated type preparation, a release-controlling agent is coated over the surface of a core. The cores may be prepared by conventional techniques known in the art such as granulation, extrusion and spheronization. Donepezil may be dispersed in the core with or without a release-controlling agent. Alternatively, donepezil may be coated onto an inert carrier to obtain the core. Further, the cores are coated with the release-controlling agent. The inert carrier may be readily available, e.g., non-pareil sugar beads or microcrystalline cellulose beads. The extended-release characteristics may be controlled in some cases by means of multiple layers of coating.

Also, coated cores may be compressed into tablets or filled into capsules or sachets.

Coating may be performed by applying one or more release controlling agent, with or without other pharmaceutically inert excipients, as a solution/suspension using any conventional coating technique known in the art, such as spray coating in a conventional coating pan or fluidized bed processor; or dip coating. The inert excipients include plasticizers such as propylene glycol, triethyl citrate, tributyl citrate, dibutyl sebacate, triacetin, polyethylene glycol, diethyl phthalate, acetylated monoglycerides, and mixtures thereof; opacifiers such as titanium dioxide, silicon dioxide, talc, calcium carbonate, behenic acid and cetyl alcohol; solvents such as water, ethanol, methanol, isopropyl alcohol, dichloromethane, acetone, or mixture thereof.

The pharmaceutical composition may further comprise other pharmaceutically acceptable excipients which include all excipients used in the art of manufacturing solid dosage forms. Examples include binders, antioxidants, diluents, lubricants/glidants, film forming polymers and coloring agents.

Specific examples of binders include methyl cellulose, hydroxypropyl cellulose (HPC-L), methylcellulose, carboxymethyl cellulose sodium, hydroxypropyl methylcellulose, polyvinylpyrrolidone, and a mixture thereof.

Specific examples of the anti-oxidant used in the present invention include ascorbic acid, sodium ascorbate, erythorbic acid, sodium erythorbate, ascorbic acid palmitate, ascorbic acid glucoside, cysteine, cysteine hydrochloride, methionine, sodium sulfite, sodium hydrogen sulfite, dibutylhydroxytoluene, butylhydroxyanisol, gallic acid derivatives, tocopherols, and a mixture thereof.

Specific examples of diluents include lactose, calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, mannitol, sorbitol, starch, starch corn, sucrose, sugar compressible, sugar confectioners, and a mixture thereof.

Coloring agents may be selected from FDA approved colorants, such as Iron Oxide, Lake of Tartrazine, Allura Red, Lake of Quinoline Yellow, Lake of Erythrosine, titanium dioxide, and a mixture thereof.

Specific examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and a mixture thereof.

The pharmaceutical composition may further be coated with non-functional layers comprising film-forming polymers, if desired.

Examples of film-forming polymers include polyvinyl pyrrolidone, hydroxypropyl methylcellulose and hydroxypropyl cellulose may be used. Alternatively, commercially available coating compositions comprising film-forming polymers marketed under various trade names, such as Opadry® may also be used for coating.

According to one embodiment, there is provided a process for the preparation of an extended-release pharmaceutical composition for an oral administration, the process includes the steps of:
a) blending donepezil with a release controlling agent and optionally, one or more pharmaceutically acceptable excipients;
b) optionally, granulating the blend of step a);
c) lubricating the blend of step a) or the granules of step b); and
d) compressing the blend of step c) into a suitably sized tablet.

According to another embodiment, there is provided a process for the preparation of an extended-release pharmaceutical composition for an oral administration, the process includes the steps of:
a) blending donepezil with a hydrophobic polymer;
b) optionally, granulating the blend of step a);
c) granulating one or more pharmaceutically acceptable excipients with a dispersion comprising a microenvironment pH modifier;
d) blending the granules of step c) with the blend of step a) or the granules of step b); and
e) compressing the blend of step d) into a suitably sized tablet.

According to another embodiment, there is provided a process for the preparation of extended-release pharmaceutical composition for an oral administration, the process including the steps of:
a) blending donepezil with a release-controlling agent and one or more pharmaceutically acceptable excipients;
b) granulating the blend of step a);
c) blending the granules of step b) with microenvironment pH modifier;
d) lubricating the blend of step c); and
e) compressing the blend of step d) into a suitably sized tablet.

Tablets may be prepared by a direct compression method or a granulation method. Granules can be prepared by dry granulation or wet granulation. Wet granulation may be carried out using granulating fluid, binder solution or hot melt of waxes. Binder solution may include a suitable hydrophilic polymer dispersed or dissolved in a solvent. Dry granulation may be carried out by roller compaction or slugging.

The solvent used for granulation and coating may be selected from water, alcohols such as methyl alcohol, ethyl alcohol or isopropyl alcohol, acetone, and mixture thereof.

The following examples illustrate the invention but do not limit the scope of the invention.

### EXAMPLES

### Example 1

| Ingredients | Qty (mg/tablet) |
|---|---|
| Intragranular | |
| Donepezil Hydrochloride | 23.00 |
| Ethyl Cellulose | 120.00 |
| Lactose Monohydrate | 83.00 |
| Hydroxyl Propyl Cellulose | 11.00 |
| Purified Water | qs |
| Extra Granular | |
| Ethyl Cellulose | 30.00 |
| Lactose Monohydrate | 30.00 |
| Fumaric Acid | 70.00 |
| Magnesium Stearate | 3.00 |
| Final Tablet Weight | 370.00 |

### Process:

1) Donepezil hydrochloride, a part of ethyl cellulose, lactose monohydrate and hydroxypropyl cellulose were blended together.
2) Blend of step 1) was granulated with purified water.
3) Granules of step 2) were blended with fumaric acid, the remaining portion of ethylcellulose and lactose monohydrate.
4) Blend of step 3) was lubricated with magnesium stearate.
5) Blend of step 4) was compressed into a suitably sized tablet.

### Dissolution Studies:

Drug release determination was carried out using HPLC method involving Kromasil C-18 column and mobile phase comprising mixture of buffer (pH 2.2) and methanol (a mixture of buffer and methanol in the ratio of 50:50).

The donepezil tablet of Example 1 was subjected to in-vitro dissolution studies. In this test, the drug-release was determined in 900 ml of 0.1 N HCL (pH 1.2), phosphate buffer (pH 6.8) and acetate buffer (4.5) using USP apparatus II with alternate sinkers and paddle speed of 50 rpm at 37° C. The dissolution profile of donepezil extended-release tablet is presented in Table 1, 2, and 3.

TABLE 1
% Drug Release of Example 1 in 900 ml of 0.1N HCL

| Time | |
|---|---|
| (hr) | % Drug release |
| 0 | 0.00 |
| 2 | 47.00 |
| 4 | 82.00 |
| 6 | 94.00 |
| 8 | 97.00 |
| 12 | 98.00 |

TABLE 2
% Drug Release Example 1 in 900 ml of Phosphate Buffer

| Time | % Drug Release |
|---|---|
| 0 | 0.00 |
| 2 | 45.00 |
| 4 | 68.00 |
| 6 | 81.00 |
| 8 | 92.00 |
| 12 | 96.00 |

TABLE 3
% Drug Release Example 1 in 900 ml of Acetate Buffer

| Time (hr) | % Drug Release |
|---|---|
| 0 | 0.00 |
| 2 | 46.00 |
| 4 | 69.00 |
| 6 | 84.00 |
| 8 | 92.00 |
| 12 | 100.00 |

The composition of Example 1 shows a pH independent release profile.

While several particular formulations have been described above, it will be apparent that various modifications and combinations of the formulations detailed in the text can be made without departing from the spirit and scope of the invention. For example, additional exemplary tablet formulations are contemplated to use various release-controlling agent in combination with various microenvironment pH modifiers.

### Example 2

| Ingredients | % w/w (wrt final tablet weight) |
|---|---|
| Donepezil Hydrochloride | 2-16% |
| Lactose | 5-50% |
| Hydroxypropyl Methylcellulose | 10-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 1-4% |
| Final Tablet Weight | 100% |

### Process:

1) Blend donepezil hydrochloride with lactose monohydrate and hydroxypropyl methylcellulose.
2) Lubricate the blend of step 1) with talc, colloidal silicon dioxide and magnesium stearate.
3) Compress the blend of step 2) into a suitably sized tablet.
4) Coat the tablet of step 3) with an aqueous dispersion of Opadry®.

### Example 3

| Ingredients | % w/w (wrt final tablet weight) |
|---|---|
| Donepezil Hydrochloride | 2-16% |
| Microcrystalline Cellulose | 5-50% |
| Polyethyleneoxide | 10-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 1-4% |
| Final Tablet Weight | 100% |

### Process:

1) Blend donepezil hydrochloride with microcrystalline cellulose and polyethylene oxide.
2) Lubricate the blend of step 1) with talc, colloidal silicon dioxide and magnesium stearate.
3) Compress the blend of step 2) into a suitably sized tablet.
4) Coat the tablet of step 3) with an aqueous dispersion of Opadry®.

### Example 4

| Ingredients | % w/w (wrt final tablet weight) |
|---|---|
| Donepezil Hydrochloride | 2-16% |
| Microcrystalline Cellulose | 5-50% |
| Hydroxypropyl Methylcellulose | 10-50% |
| Hydroxy Propyl Cellulose (HPC-L) | |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 1-4% |
| Final Tablet Weight | 100% |

### Process:

1) Blend donepezil hydrochloride with microcrystalline cellulose and hydroxypropyl methylcellulose.
2) Granulate the blend of step 1) with a non-aqueous solution of hydroxy propyl cellulose (HPC-L).
3) Lubricate the granules of step 2) with talc, colloidal silicon dioxide and magnesium stearate.
4) Compress the blend of step 3) into a suitably sized tablet.
5) Coat the tablet of step 4) with an aqueous dispersion of Opadry®.

### Example 5

| Ingredients | % w/w (wrt final tablet weight) |
|---|---|
| Donepezil hydrochloride | 2-16% |
| Microcrystalline cellulose | 5-50% |
| Polyethylene oxide | 10-50% |
| Colloidal silicon dioxide | 0.5-2% |
| Talc | 0.1-2% |
| Magnesium Stearate | 0.5-2% |

Opadry ® Coating

| | |
|---|---|
| Opadry ® | 1-4% |
| Final tablet weight | 100% |

### Process:

1) Blend donepezil hydrochloride with microcrystalline cellulose and a part of colloidal silicon dioxide.
2) Compact the blend of step 1) to form granules.
3) Blend the granules of step 2) with polyethylene oxide.
4) Lubricate the granules of step 3) with talc, remaining part of colloidal silicon dioxide and magnesium stearate.
5) Compress the blend of step 4) into a suitably sized tablet.
6) Coat the tablet of step 5) with an aqueous dispersion of Opadry®.

### Example 6

| Ingredients | % w/w (wrt final tablet weight) |
|---|---|
| Donepezil Hydrochloride | 2-16% |
| Lactose | 5-50% |
| Hydroxyl Ethyl Cellulose | 10-50% |
| Fumaric Acid | 1-10% |
| Colloidal Silicon Dioxide | 0.1-2% |
| Talc | 0.1-2% |
| Magnesium Stearate | 0.5-2% |

Opadry Coating

| | |
|---|---|
| Opadry ® | 1-4% |
| Final Tablet Weight | 100% |

### Process:

1) Granulate lactose with aqueous solution of fumaric acid
2) Blend the granules of step 1) with donepezil hydrochloride and hydroxylethyl cellulose.
3) Lubricate the blend of step 2) with talc, colloidal silicon dioxide and magnesium stearate.
4) Compress the blend of step 3) into a suitably sized tablet.
5) Coat the tablet of step 4) with an aqueous dispersion of Opadry®.

### Example 7

| Ingredients | % w/w (wrt final tablet weight) |
|---|---|
| Donepezil hydrochloride | 2-16% |
| Microcrystalline cellulose | 5-50% |
| Hydroxylpropyl methylcellulose | 10-50% |
| Ascorbic acid | 1-10% |
| Colloidal silicon dioxide | 0.1-2% |
| Talc | 0.1-2% |
| Magnesium Stearate | 0.5-2% |

Opadry ® Coating

| | |
|---|---|
| Opadry ® | 1-4% |
| Final Tablet Weight | 100% |

### Process:

1) Blend donepezil hydrochloride with microcrystalline cellulose, ascorbic acid and hydroxypropyl methylcellulose
2) Lubricate the blend of step 1) with talc, colloidal silicon dioxide and magnesium stearate.
3) Compress the blend of step 2) into a suitably sized tablet.
4) Coat the tablet of step 3) with an aqueous dispersion of Opadry®.

### Example 8

| Ingredients | % w/w (wrt final tablet weight) |
|---|---|
| Donepezil Hydrochloride | 2-16% |
| Lactose | 5-50% |
| Cornstarch | 5-15% |
| Ethyl Cellulose | 10-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 2-6% |
| Final Tablet Weight | 100% |

### Process:

1) Blend donepezil hydrochloride with lactose, corn starch and ethyl cellulose.
2) Lubricate the blend of step 1) with talc, colloidal silicon dioxide and magnesium stearate.
3) Compress the blend of step 2) into a suitably sized tablet.
4) Coat the tablet of step 3) with an aqueous dispersion of Opadry®.

### Example 9

| Ingredients | % w/w (wrt final tablet weight) |
|---|---|
| Donepezil Hydrochloride | 2-16% |
| Lactose | 5-50% |
| Eudragit ® RL | 10-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 2-6% |
| Final Tablet Weight | 100% |

### Process:

1) Granulate donepezil with an aqueous dispersion of Eudragit®RL.
2) Blend the granules of step 1) with lactose.
3) Lubricate the blend of step 2) with talc, colloidal silicon dioxide and magnesium stearate.
4) Compress the blend of step 3) into a suitable size tablet.
5) Coat the tablet of step 4) with an aqueous dispersion of Opadry®.

### Example 10

| Ingredients | % w/w (wrt final tablet weight) |
|---|---|
| Donepezil Hydrochloride | 2-16% |
| Microcrystalline Cellulose | 5-50% |
| Ethyl Cellulose | 10-50% |
| Fumaric Acid | 1-10% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 2-6% |
| Final Tablet Weight | 100% |

### Process:

1) Granulate microcrystalline cellulose with an aqueous solution of fumaric acid
2) Blend the granules of step 1) with donepezil hydrochloride and ethyl cellulose.
3) Lubricate the blend of step 2) with talc, colloidal silicon dioxide and magnesium stearate.
4) Compress the blend of step 3) into a suitably sized tablet.
5) Coat the tablet of step 4) with an aqueous dispersion of Opadry®.

### Example 11

| Ingredients | % w/w (wrt final tablet weight) |
|---|---|
| Donepezil Hydrochloride | 2-16% |
| Microcrystalline Cellulose | 5-50% |
| Eudragit ® RS | 10-50% |
| Ascorbic Acid | 1-10% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 2-6% |
| Final Tablet Weight | 100% |

### Process:

1) Granulate donepezil hydrochloride with an aqueous dispersion of Eudragit®RS.
2) Granulate microcrystalline cellulose with an aqueous solution of ascorbic acid.
3) Blend the granules of step 1) and step 2) together.
4) Lubricate the granules of step 3) with talc, colloidal silicon dioxide and magnesium stearate.
5) Compress the blend of step 4) into a suitably sized tablet.
6) Coat the tablet of step 5) with an aqueous dispersion of Opadry®.

### Example 12

| Ingredients | % w/w (wrt final tablet weight) |
|---|---|
| Donepezil Hydrochloride | 2-16% |
| Microcrystalline Cellulose | 5-50% |
| Ethyl Cellulose | 10-50% |
| Hydroxypropyl Methylcellulose | 10-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 2-6% |
| Final Tablet Weight | 100% |

### Process:

1) Blend donepezil hydrochloride with microcrystalline cellulose, ethyl cellulose, and hydroxypropyl methylcellulose.
2) Lubricate the blend of step 1) with talc, colloidal silicon dioxide and magnesium stearate.
3) Compress the blend of step 2) into a suitably sized tablet.
4) Coat the tablet of step 3) with an aqueous dispersion of Opadry®.

### Example 13

| Ingredients | % w/w (wrt final tablet weight) |
|---|---|
| Donepezil Hydrochloride | 2-16% |
| Microcrystalline Cellulose | 5-50% |
| Eudragit ® RSPO | 10-50% |
| Hydroxy Propyl Cellulose | 10-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 2-6% |
| Final Tablet Weight | 100% |

### Process:

1) Blend donepezil hydrochloride with Eudragit®RSPO, hydroxy propyl cellulose and microcrystalline cellulose together.
2) Lubricate the blend of step 1) with talc, colloidal silicon dioxide and magnesium stearate.
3) Compress the blend of step 2) into a suitably sized tablet.
4) Coat the tablet of step 3) with an aqueous dispersion of Opadry®.

### Example 14

| Ingredients | % w/w (wrt final tablet weight) |
|---|---|
| Donepezil Hydrochloride | 2-16% |
| Microcrystalline Cellulose | 5-50% |
| Glyceryl Behanate | 10-50% |
| Polyethylene Oxide | 10-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 2-6% |
| Final Tablet Weight | 100% |

### Process:

1) Blend donepezil hydrochloride with glyceryl behanate, polyethylene oxide, and microcrystalline cellulose.
2) Lubricate the blend of step 1) with talc, colloidal silicon dioxide and magnesium stearate.
3) Compress the blend of step 2) into a suitably sized tablet.
4) Coat the tablet of step 3) with an aqueous dispersion of Opadry®.

### Example 15

| Ingredients | % w/w (wrt final tablet weight) |
|---|---|
| Donepezil Hydrochloride | 2-16% |
| Lactose | 5-50% |
| Ethyl Cellulose | 10-50% |
| Ascorbic Acid | 1-10% |
| Hydroxypropyl Cellulose | 10-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 2-6% |
| Final Tablet Weight | 100% |

### Process:

1) Blend donepezil hydrochloride with ethyl cellulose, ascorbic acid and hydroxypropyl cellulose.
2) Lubricate blend of step 1) with talc, magnesium stearate and colloidal silicon dioxide.
3) Compress the blend of step 2) into a suitably sized tablet.
4) Coat the tablet of step 3) with an aqueous dispersion of Opadry®.

### Example 16

| Ingredients | % w/w (wrt final tablet weight) |
|---|---|
| Donepezil Hydrochloride | 2-16% |
| Microcrystalline Cellulose | 5-50% |
| Hydrogenated Castor Oil | 10-50% |
| Phosphoric Acid | 1-10% |
| Xanthan Gum | 10-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 2-6% |
| Final Tablet Weight | 100% |

### Process:

1) Melt granulate donepezil hydrochloride, microcrystalline cellulose with hydrogenated castor oil.
2) Blend the granules of step 1) with xanthan gum.
3) Lubricate the blend of step 2) with talc, colloidal silicon dioxide and magnesium stearate.
4) Compress the lubricated blend of step 3) into a suitably sized tablet.
5) Coat the tablet of step 4) with an aqueous dispersion of Opadry®.

### Example 17

| Ingredients | % w/w (wrt final tablet weight) |
|---|---|
| Donepezil Hydrochloride | 2-16% |
| Microcrystalline Cellulose | 5-50% |
| Carnauba Wax | 10-50% |
| Glycine Hydrochloride | 0.1-1% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 1-4% |
| Final Tablet Weight | 100% |

### Process:

1) Disperse donepezil hydrochloride in a hot melt of carnauba wax to form granules.
2) Granulate microcrystalline cellulose with an alcoholic solution of glycine hydrochloride.
3) Blend the granules of step 1) and step 2).
4) Lubricate the blend of step 3) with talc, colloidal silicon dioxide and magnesium stearate.
5) Compress the blend of step 4) into a suitably sized tablet.
6) Coat the tablet of step 5) with an aqueous dispersion of Opadry®.

### Example 18

| Ingredients | % w/w (wrt final tablet weight) |
|---|---|
| Donepezil Hydrochloride | 2-16% |
| Lactose | 5-50% |
| Hydroxypropyl Methylcellulose | 10-50% |
| Polyvinyl Pyrrolidone | 1-10% |
| Malic Acid | 0.1-1% |
| Colloidal Silicon Dioxide | 0.1-2% |
| Talc | 0.1-2% |

Extended Release coating

| | |
|---|---|
| Eudragit RS | 5-10% |
| PEG | 0.5-3% |

Opadry ® Coating

| | |
|---|---|
| Opadry ® | 1-4% |
| Final tablet weight | 100% |

### Process:

1) Granulate lactose and polyvinyl pyrrolidone with aqueous solution of malic acid.
2) Blend donepezil hydrochloride and hydroxypropyl methylcellulose with granules of step 1).
3) Lubricate the blend of step 2) with colloidal silicon dioxide and talc.
4) Compress the blend of step 3) into a suitably sized tablet.
5) Coat the tablet of step 4) using a non aqueous solution of Eudragit®RS and polyethylene glycol.
6) Coat the coated tablets of step 5) with an aqueous dispersion of Opadry®.

### Example 19

| Ingredients | % w/w (wrt final tablet weight) |
|---|---|
| Donepezil Hydrochloride | 2-16% |
| Microcrystalline Cellulose | 5-50% |
| Carbopol | 10-50% |
| Polyvinyl Pyrrolidone | 1-10% |
| Colloidal Silicon Dioxide | 0.1-2% |
| Talc | 0.1-2% |

Extended Release Coating

| | |
|---|---|
| Ethyl Cellulose | 5-10% |
| Peg | 0.5-3% |

Opadry ® Coating

| | |
|---|---|
| Opadry ® | 1-4% |
| Final Tablet Weight | 100% |

### Process:

1) Blend donepezil hydrochloride with microcrystalline cellulose, carbopol and polyvinyl pyrrolidone together.
2) Lubricate the blend of step 1) with colloidal silicon dioxide and talc.
3) Compress the lubricated blend of step 2) into a tablet.
4) Coat the tablet of step 3) with non-aqueous dispersion of ethyl cellulose and polyethylene glycol.
5) Coat the coated tablets of step 4) with an aqueous dispersion of Opadry®.

### Example 20

| Ingredients | % w/w (wrt final tablet weight) |
|---|---|
| Donepezil Hydrochloride | 2-16% |
| Lactose | 5-50% |
| Hydroxypropyl Methylcellulose | 10-50% |
| Eudragit ® L-10055 | 1-50% |
| L-Cysteine Hcl | 0.5-2% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 1-4% |
| Final Tablet Weight | 100% |

### Process:

1) Blend donepezil hydrochloride with lactose, hydroxypropyl methylcellulose, L-cysteine HCl and Eudragit®L-10055 together.
2) Lubricate the blend of step 1) with colloidal silicon dioxide, talc and magnesium stearate.
3) Compress the blend of step 2) into a suitably sized tablet.
4) Coat the tablets of step 3) using an aqueous dispersion of Opadry®.

### Example 21

| Ingredients | % w/w (wrt final tablet weight) |
|---|---|
| Donepezil Hydrochloride | 2-16% |
| Microcrystalline Cellulose | 5-50% |
| Polyethylene Oxide | 10-50% |
| Hydroxypropyl Methylcellulose | 1-50% |
| Phthalate | |
| Colloidal Silicon Dioxide | 0.1-2% |
| Talc | 0.1-2% |
| Magnesium Stearate | 0.5-2% |

Enteric Coating

| | |
|---|---|
| Eudragit ® L-10055 | 1-5% |
| Polyethylene Glycol | 0.1-1% |
| Final Tablet Weight | 100% |

### Process:

1) Blend donepezil hydrochloride with microcrystalline cellulose and polyethylene oxide and hydroxypropyl methylcellulose phthalate.
2) Lubricate the blend of step 1) with colloidal silicon dioxide, magnesium stearate and talc.
3) Compress the lubricated blend of step 2) with a suitably sized tablet.
4) Coat the tablets of step 3) using an alcoholic dispersion of Eudragit®L-10055 and polyethylene glycol.

### Example 22

| Ingredients | % w/w (wrt final tablet weight) |
|---|---|
| Donepezil Hydrochloride | 2-16% |
| Lactose | 5-50% |
| Hydrogenated Castor Oil | 10-50% |
| Eugradit ® L-10055 | 1-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 1-4% |
| Final Tablet Weight | 100% |

### Process:

1) Disperse donepezil hydrochloride in a hot melt of hydrogenated castor oil to form granules.
2) Blend the granules of step 1) with lactose and Eugradit®L-10055.
3) Lubricate the blend of step 2) with talc, colloidal silicon dioxide, and magnesium stearate.
4) Compress the lubricated blend of step 3) into a suitably sized tablet.
5) Coat the tablet of step 4) with an aqueous dispersion of Opadry®.

### Example 23

| Ingredients | % w/w (wrt final tablet weight) |
|---|---|
| Donepezil Hydrochloride | 2-16% |
| Mannitol | 5-50% |
| Glyceryl Behanate | 10-50% |
| Colloidal Silicon Dioxide | 0.1-2% |
| Talc | 0.1-2% |

Extended Release Coating

| | |
|---|---|
| HPMC Phallate | 5-10% |
| Triethyl Citrate | 0.5-3% |
| Silicon Dioxide | 2-5% |

Opadry ® Coating

| | |
|---|---|
| Opadry ® | 1-4% |
| Final Tablet Weight | 100% |

### Process:

1) Disperse donepezil hydrochloride in a hot melt of glyceryl behenate to form granules.
2) Blend the granules of step 1) with mannitol.
3) Lubricate the blend of step 2) with colloidal silicon dioxide and talc.
4) Compress the blend of step 3) into a suitably sized tablet.
5) Coat the tablets of step 4) using an alcoholic dispersion of HPMC phthalate, triethyl citrate and silicon dioxide together.
6) Coat the coated tablet of step 5) with an aqueous dispersion of Opadry®.

### Example 24

| Ingredients | % w/w (wrt final tablet weight) |
|---|---|
| Donepezil Hydrochloride | 2-16% |
| Lactose | 5-50% |
| Ethyl Cellulose | 10-50% |
| Bht | 0.01-0.1% |
| Colloidal Silicon Dioxide | 0.1-2% |
| Talc | 0.1-2% |

Extended Release Coating

| | |
|---|---|
| Eudragit ® L-10055 | 5-10% |
| Triethyl Citrate | 0.5-3% |
| Silicon Dioxide | 2-5% |

Opadry ® Coating

| | |
|---|---|
| Opadry ® | 1-4% |
| Final Tablet Weight | 100% |

### Process:

1) Blend donepezil hydrochloride with lactose, BHT and ethylcellulose together.
2) Lubricate the blend of step 1) with colloidal silicon dioxide and talc.
3) Compress the blend of step 2) into a suitably sized tablet.
4) Coat the tablets of step 3) using an alcoholic dispersion of Eudragit®L-10055, triethyl citrate, silicon dioxide together.
5) Coat the coated tablet of step 4) into a suitably sized tablet.

### Example 25

| Ingredients | % w/w (wrt final tablet weight) |
|---|---|
| Donepezil Hydrochloride | 2-16% |
| Lactose | 5-50% |
| Eudragit ® L-10055 | 10-50% |
| Colloidal Silicon Dioxide | 0.1-2% |
| Talc | 0.1-2% |

Extended Release Coating

| | |
|---|---|
| Ethyl Cellulose | 5-10% |
| Peg | 0.5-3% |

Opadry ® Coating

| | |
|---|---|
| Opadry ® | 1-4% |
| Final Tablet Weight | 100% |

### Process:

1) Blend donepezil hydrochloride with lactose and Eudragit®L-10055 together.
2) Lubricate the blend of step 1) with colloidal silicon dioxide and talc.
3) Compress the lubricated blend of step 2) into a suitably sized tablet.
4) Coat the tablets of step 3) using a non aqueous dispersion of ethyl cellulose and polyethylene glycol together.
5) Coat the coated tablets of step 4) with an aqueous dispersion of Opadry®.

### Example 26

| Ingredients | % w/w (wrt final tablet weight) |
|---|---|
| Donepezil Hydrochloride | 2-16% |
| Mannitol | 5-50% |
| Hydroxypropyl Methylcellulose | 10-50% |
| Phthalate | |
| Ascorbic Acid | 1-10% |
| Colloidal Silicon Dioxide | 0.1-2% |
| Talc | 0.1-2% |

Extended Release Coating

| | |
|---|---|
| Eudragit ® RS | 5-10% |
| Triethyl Citrate | 0.5-3% |

Opadry ® Coating

| | |
|---|---|
| Opadry ® | 1-4% |
| Final Tablet Weight | 100% |

### Process:

1) Blend donepezil hydrochloride with mannitol, ascorbic acid and hydroxypropyl methylcellulose phthalate together.
2) Lubricate the blend of step 1) with colloidal silicon dioxide and talc.
3) Compress the lubricated blend of step 2) into a suitably sized tablet.
4) Coat the tablet of step 3) using a non aqueous solution of Eudragit®RS and triethyl citrate.
5) Coat the coated tablets of step 4) with an aqueous dispersion of Opadry®.

### Example 27

| Ingredients | % w/w (wrt final tablet weight) |
|---|---|
| Donepezil Hydrochloride | 2-16% |
| Microcrystalline Cellulose | 5-50% |
| Eudragit ® L30D-55 | |

Extended Release Coating

| | |
|---|---|
| Ethyl Cellulose | 5-10% |
| Triethyl Citrate | 0.5-3% |

Tablet Excipients

| | |
|---|---|
| Microcrystalline Cellulose | 20-50% |
| Colloidal Silicon Dioxide | 2-5% |
| Opadry ® | 2-4% |
| Final Tablet Weight | 100% |

### Process:

1) Granulate donepezil hydrochloride, microcrystalline cellulose using an aqueous dispersion of Eudragit®L30D-55.
2) Extrude the wet mass of step 1) to form suitably sized pellets.
3) Coat the pellets of step 2) with ethyl cellulose and triethyl cutrate.
4) Blend the coated pellets of step 3) with microcrystalline cellulose and colloidal silicon dioxide and compress into tablets of a suitably sized tablet.
5) Coat the coated tablets of step 4) with an aqueous dispersion of Opadry®.

### Example 28

| Ingredients | % w/w (wrt final tablet weight) |
|---|---|
| Donepezil Hydrochloride | 2-16% |
| Sugar Spheres | 5-50% |
| Hydroxypropyl Methylcellulose | 10-25% |

Extended Release Coating

| | |
|---|---|
| Ethyl Cellulose | 5-10% |
| Triethyl Citrate | 0.5-3% |

Eudragit ® Coating

| | |
|---|---|
| Eudragit ® L30D-55 | 5-10% |
| Triethyl Citrate | 0.5-5% |

Tablet Excipients

| | |
|---|---|
| Microcrystalline Cellulose | 20-50% |
| Colloidal Silicon Dioxide | 2-5% |
| Opadry ® | 2-4% |
| Final Tablet Weight | 100% |

### Process:

1) Coat aqueous suspension of donepezil and hydroxypropyl methylcellulose onto sugar spheres.
2) Coat the coated sphere of step 1) with ethyl cellulose and triethyl citrate.
3) Coat the coated sphere of step 2) with Eudragit®L30D-55 and triethyl citrate.
4) Blend the coated sphere of step 3) with microcrystalline cellulose and colloidal silicon dioxide and compress into tablets of a suitable size.
5) Coat the tablets of step 4) with an aqueous dispersion of Opadry®.

### Example 29

| Ingredients | % w/w (wrt final tablet weight) |
|---|---|
| Donepezil Hydrochloride | 2-16% |
| Lactose | 5-50% |
| Hydroxypropyl Cellulose | 10-50% |
| Eudragit ® L-10055 | 1-50% |
| Colloidal Silicon Dioxide | 0.1-2% |
| Talc | 0.1-2% |
| Magnesium Stearate | 0.5-2% |

Opadry ® Coating

| | |
|---|---|
| Opadry ® | 1-4% |
| Final Tablet Weight | 100% |

### Process:

1) Blend donepezil hydrochloride with lactose, hydroxypropyl cellulose and Eudragit®L-10055 together.
2) Lubricate the blend of step 1) with colloidal silicon dioxide, talc and magnesium stearate.
3) Compress the blend of step 2) into a suitably sized tablet.
4) Coat the tablets of step 3) using an aqueous dispersion of Opadry®.

1. An extended-release pharmaceutical composition for an oral administration consisting essentially of:
a) donepezil or a pharmaceutically acceptable salt thereof;
b) a release-controlling agent selected from the group consisting of hydrophilic polymer, hydrophobic material, hydrophobic polymer, and a mixture thereof; and
c) a microenvironment pH modifier.

2. The extended-release pharmaceutical composition of claim 1, wherein the hydrophobic polymer is selected from the group consisting of ethylcellulose, cellulose acetate, cellulose propionate, cellulose butyrate, methacrylic acid-acrylic acid copolymers, and a mixture thereof.

3. The extended-release pharmaceutical composition of claim 1, wherein the hydrophilic polymer is selected from the group consisting of polyvinylpyrrolidone, hydroxypropylcellulose, methylcellulose, hydroxypropylmethyl cellulose, polyethylene oxide, acrylic acid copolymers, and a mixture thereof.

4. The extended-release pharmaceutical composition of claim 1, wherein the hydrophobic material is selected from the group consisting of hydrogenated vegetable oil, hydrogenated castor oil, carnauba wax, candellia wax, beeswax, paraffin wax, stearic acid, glyceryl behenate, cetyl alcohol, cetostearyl alcohol, and a mixture thereof.

5. The extended-release pharmaceutical composition of claim 1, wherein the microenvironment pH modifier is selected form the group consisting of inorganic acid, amino acid, organic acid, and a mixture thereof.

6. The extended-release pharmaceutical composition of claim 5, wherein the microenvironment pH modifier is an organic acid selected from the group consisting of lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid and salicylic acid; tosylic acid, mesylic acid, malic acid, and a mixture thereof.

7. The extended-release pharmaceutical composition of claim 1, wherein the microenvironment pH modifier is fumaric acid and hydrophobic polymer is ethyl cellulose.

8. The extended-release pharmaceutical composition of claim 1, wherein the pharmaceutical composition is in the form of tablet, capsules or granules.

9. The extended-release pharmaceutical composition of claim 8, wherein the tablet is in the form of matrix.

10. The extended-release pharmaceutical composition of claim 1, wherein said pharmaceutical composition does not comprise enteric polymer.
